(19) United States
(12) Patent Application Publication
(10) Pub. No.: US 2006/0241777 A1
(43) Pub. Date: Oct. 26, 2006

(54) Resorbable medical implants and methods

(76) Inventors: Jason Ian Partin, Encinitas, CA (US); Robert J. Ball, San Marcos, CA (US); Arnold-Peter C. Weiss, Barrington, RI (US); Daniel Herren, Lengghalde 2, Zurich (CH); Peter Hoepfner, Glenview, IL (US)

Correspondence Address: Jenkins, Wilson & Taylor, P.A.;University Tower, Suite 1400, 3100 Tower Boulevard, Durham, NC, 27707, US

(21) Appl. No.: 11/291,711

(22) Filed: Nov. 30, 2005

Related U.S. Application Data
(60) Provisional application No. 60/631,689, filed on Nov. 30, 2004.

(51) Int. Cl. A61F 2/42; A61F 2/28

(52) U.S. Cl. 623/21.11; 623/23.5

(57) ABSTRACT

Resorbable medical implants and related methods are disclosed for placement against bone and/or for filling a void such as a void between bones. The resorbable medical implants can be structured and configured as desired, for example, to conform to surfaces of adjacent bones and/or to conform to a desired bone shape for replacement otherwise. Short-term fixation of the resorbable medical implants can advantageously assist in promoting and allowing growth of new bone tissue into the implant as the implant gradually resorbs. The implant can comprise a material that is porous with microscopic and non-microscopic pores. In one aspect, the resorbable implant can be shaped and used as a trapezium bone replacement.

### RELATED APPLICATION

This application claims the benefit of commonly owned U.S. Provisional Patent Application Ser. No. 60/631,689, entitled “Methods and Devices To Fill Voids From Surgically Removed Or Damaged Bones”, filed Nov. 30, 2004, the disclosure of which is incorporated herein by reference in its entirety.

### TECHNICAL FIELD

The presently disclosed subject matter relates generally to orthopedic apparatuses and methods, and more particularly to resorbable medical implants and methods.

### BACKGROUND

A variety of medical situations can benefit from small bone replacement and implant material placement. Clinical situations where small-bone replacement can be desirable can include, for example, treatment of carpometacarpal (CMC) or basal joint arthritis where the trapezium bone is removed, severe injuries of the scaphoid bone, and corpectomies for Keinbock's disease. While a variety of materials and techniques exist for providing implant material such as for bone replacement and for promoting and facilitating new bone tissue in growth, room for improvement remains in such materials and techniques.

Treatment of CMC arthritis in particular is the second most common procedure in hand surgery. A common treatment is to remove the trapezium bone, pin the metacarpal bone in place for initial stability, and insert a tendon graft into the space created by removal of the trapezium. Because of its design, the basal joint has a tendency to wear out and develop arthritis early in life. The trapezium bone at the base of the thumb has joint connections to three other bones, and any of these bones and the trapezium can develop arthritis on their surfaces. Basal joint stability is maintained by the contours of the surfaces of the bones comprising the joint as well as the surrounding ligaments and muscles. Disruption of the joint surfaces or the supporting ligaments associated with the basal joint can lead to subluxation as well pain and swelling. The ends of the bones adjacent the trapezium are normally covered with articular cartilage, which provides a slippery surface and can act as a cushion while allowing the bones to move freely. Arthritis can destroy the articular surfaces and cause the joint to become painfully inflamed.

Where surgery is necessary for joint problems, a variety of techniques can be used, including, for example, removal of damaged joint surfaces or damaged bones themselves and creation of a new, substitute joint. This procedure is known as arthroplasty. Interposition arthroplasty is a commonly know procedure where damaged or degenerated joint surfaces are suitably removed and material is placed into position, or interposed, between the bones. The interposed material can serve as a cushion to prevent impingement and collapse of adjacent bones into a void.

In addition to the surgical treatment mentioned above that has been used to treat basal joint arthritis, a variety of prosthetic materials have also been used. One such implant is disclosed in U.S. Pat. No. 6,017,366 for resorbable, interposition arthroplasty implant is disclosed. Problems that have been common with the use of prosthetic implants, however, include migration of an interposed implant.

### SUMMARY

As disclosed herein, novel resorbable medical implants and methods are provided in accordance with the present subject matter.

An object of the present disclosure is to provide novel and improved resorbable medical implants and methods. An object having been stated, and which is achieved in whole or in part by the present subject matter, other objects will become evident as the description proceeds when taken in connection with the accompanying drawings as best described hereinbelow.

### BRIEF DESCRIPTION OF THE DRAWINGS

FIG. 1A of the drawings is a perspective view of one form of a resorbable medical implant according to the present disclosure;

FIG. 1B of the drawings is a close-up view of a portion of the resorbable medical implant of FIG. 1A showing microscopic pores;

FIG. 2A of the drawings is a right side elevation view of the resorbable medical implant shown in FIG. 1A;

FIG. 2B of the drawings is a left side elevation view of the resorbable medical implant shown in FIG. 1A;

FIG. 3 of the drawings is a cross sectional view drawn along line A-A of FIG. 2A;

FIG. 4 of the drawings is an end view of the resorbable medical implant shown in FIG. 1A;

FIG. 5 of the drawings is a posterior view of the palm bones of a hand:

FIG. 6 of the drawings is a posterior view of the palm bones of the hand with a replacement trapezium bone in place;

FIG. 7 of the drawings is a close-up view of a portion of the surface of the resorbable medical implant of FIG. 1A against adjacent bone and with sutures securing them together; and

FIG. 8 of the drawings is a cross sectional view similar to that in FIG. 7 with a portion of the surface of the resorbable medical implant against adjacent bone with an a resorbable plug securing them together.

### DETAILED DESCRIPTION

In accordance with the present disclosure, novel resorbable medical implants and methods are provided. Implants and related methods according to the present subject matter can be used in a variety of medical situations in a variety of manners and for a variety of purposes such as disclosed for example herein and as can be appreciated by those of skill in the art. Implants and methods according to the present subject matter can be used, for example, for placement of an implant against bone for promoting and facilitating growth of new bone tissue. This use can be particularly advantageous for filling in voids between bones and for replacing damaged or degenerated bones or bone surfaces.

Implants and related methods according to the subject matter disclosed herein provide a resorbable medical implant that can be placed against one or more bones or simply placed in a void with or without contact with bone. When used for filling a bone void, an implant according to the present subject matter can allow effective maintenance of a space between bones and therefore prevent impingement of the bones. As described further herein, short-term fixation of an implant according to the present disclosure can advantageously assist in promoting and allowing growth of new bone tissue into the implant as the implant gradually resorbs.

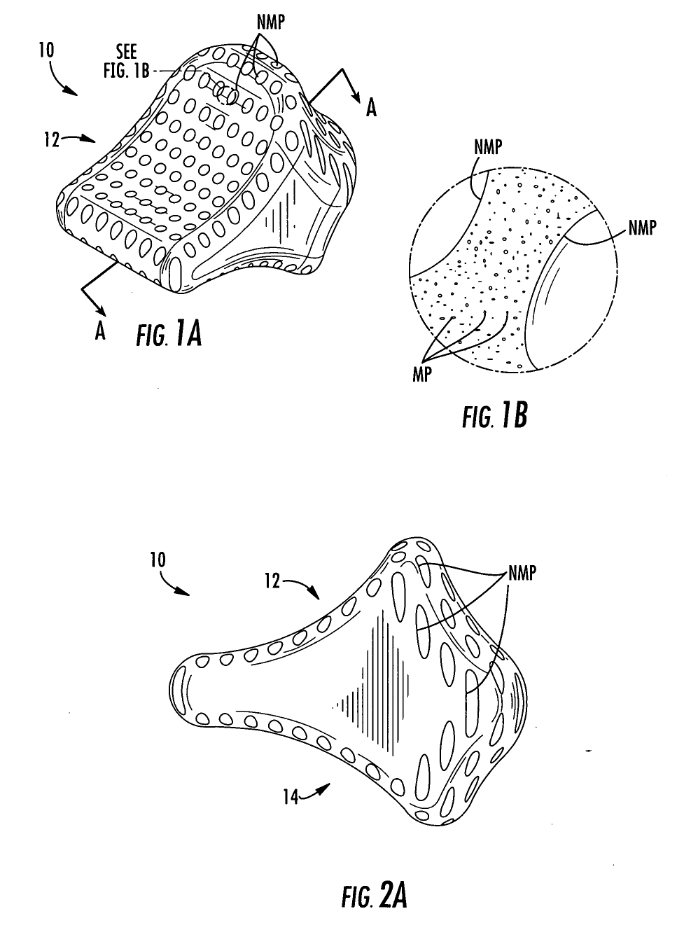

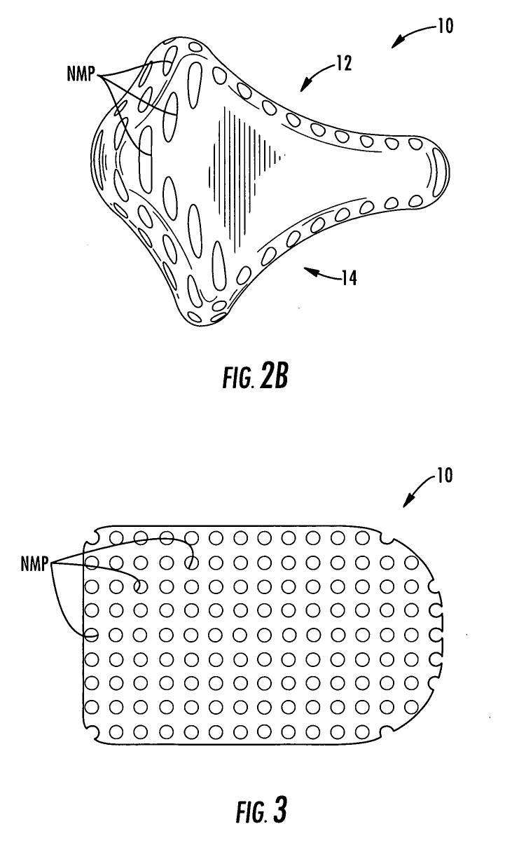

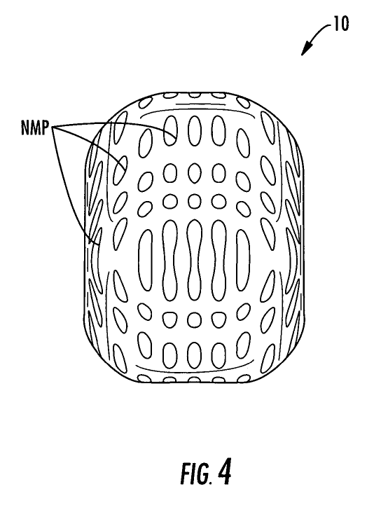

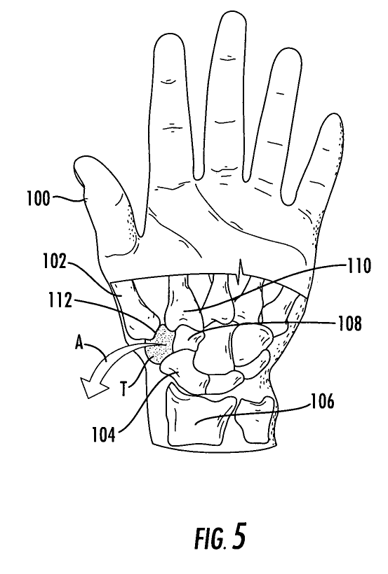

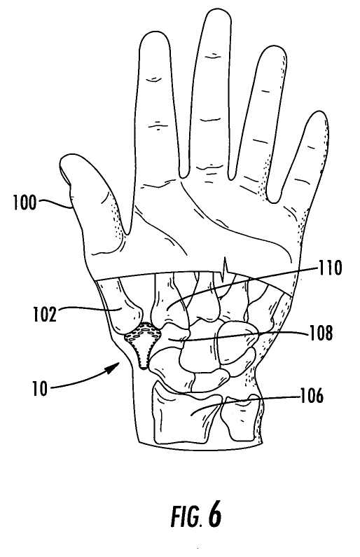

Referring to FIG. 1A of the drawings, a perspective view of an example of one embodiment of a resorbable medical implant according to the present disclosure is shown as resorbable medical implant generally designated 10 which is a structural article for orthopedic use. Resorbable medical implant 10 is shown in FIG. 1 similar to the shape of a natural trapezium bone T (shown in FIG. 5). As shown in FIGS. 1-4, resorbable implant 10 can have saddle-shaped surfaces such as for example saddle-shaped surfaces generally designated 12 and 14 rather than just one, upper saddle-shaped surface as with a natural trapezium. The existence of more than one saddle-shaped surface allows resorbable implant 10 to be used for either a right hand or left hand trapezium replacement. It is also envisioned that resorbable implant 10 could be formed to be identical to the shape of a natural trapezium bone. While resorbable implant 10 is shown in a form suitable for replacement of a trapezium bone such as trapezium bone T, a resorbable implant according to this disclosure can be formed or manufactured into any suitable, predetermined configuration and used for purposes other than trapezium replacement, such as for other purposes where interposition of a resorbable implant or simply placement of a resorbable implant against bone in other areas may be desirable. Other anatomical sites can include but are not limited to other carpal bones, wrist bones, and elbow bones as well as the small joints of the foot.

Resorbable implant 10 according to the present subject matter can be made of any suitable material known now or later developed. The implant material can comprise synthetic, zenograft, or allograft material. Known resorbable materials can include, for example and without limitation, copolymers of lactic acid and/or glycolic acid (PLLA/PLGA). Other materials can, for example, comprise polyglycolide: trimethylene carbonate (PGA:TMC), collagen, tricalcium phosphate, hydroxyapatite, hydrogels, or combinations thereof. The proportions of these or any other materials can as possible be adjusted to achieve a desired resorption rate as known to those of skill in the art. Resorbable implant 10 can be entirely resorbable.

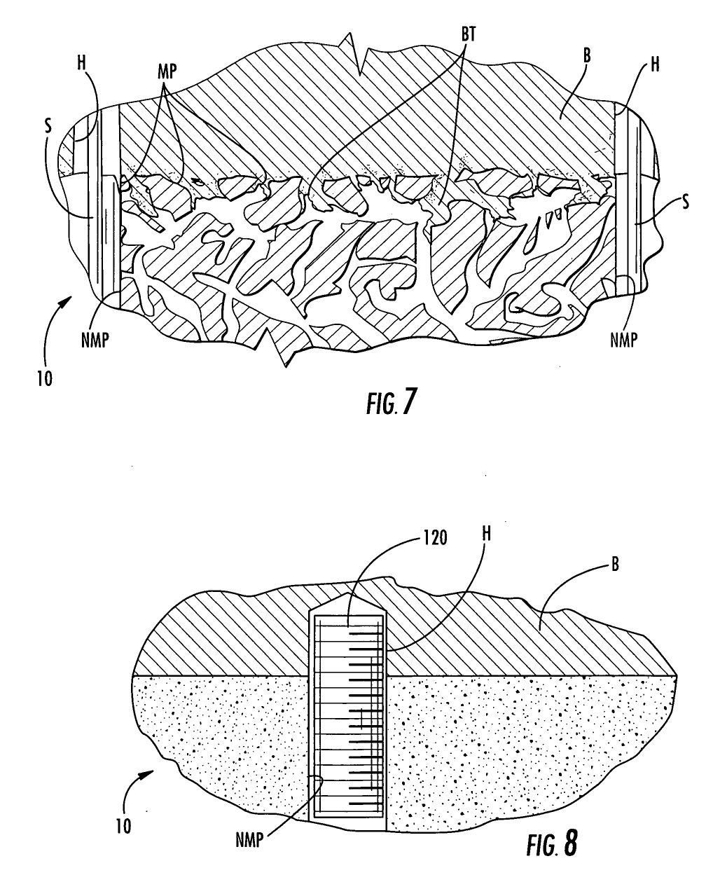

In accordance with the present disclosure, resorbable implant 10 can be formed of a material that is porous. The material used to form resorbable implant 10 can be manufactured in any suitable manner to have a porous form such as an open-pore foam structure. Resorbable implant 10 can be manufactured using conventional manufacturing techniques to comprise pores that are microscopic in diametrical size, as shown for example by microscopic pores MP in the close-up view of resorbable implant 10 shown in FIG. 1A. Microscopic pores MP can provide a network of interconnected porosities within resorbable implant 10 into which bone tissue can grow. As can be appreciated by those of skill in the art, microscopic pores MP can promote and facilitate ingrowth of new bone tissue and are particularly suitable and advantageous when resorbable implant 10 is placed against a surface of a bone as described further with respect to FIG. 7. The microscopic porosity can be any suitable amount of porosity such as, for example and without limitation, an amount wherein the porosity percentage is from about 50% to about 99%. Microscopic pores MP can be of any suitable microscopic size, which is generally considered to be something measure in terms of thousands of a millimeter. Microscopic pores MP can therefore be referred to for size by a measurement in terms of micrometers or microns, as one micron (um) is equal to one-millionth of a meter, which is equal to one-thousandth of a millimeter (mm) (1000 um=1 mm). For example and without limitation, microscopic pores MP can be of any diametrical size up to about 200 microns.

In addition to the microscopic pores, resorbable implant 10 can comprise non-microscopic pores such as for example non-microscopic pores NMP on resorbable implant 10 shown best in FIGS. 1A-4. Non-microscopic pores NMP can also be referred to as macroscopic as they are not of a size that is typically considered microscopic. Non-microscopic pores NMP are instead of a scale larger in diametrical size from microscopic pores MP. For example and without limitation, non-microscopic pores NMP can be at least a size of about 0.5 millimeters or larger in diameter. Non-microscopic pores NMP can also range, for example, in size from about 0.5 millimeters to about 2.5 millimeters. Non-microscopic pores NMP can be drilled into resorbable implant 10 or any other suitable technique can be used to create non-microscopic pores NMP. In use of resorbable implant 10, and as described further herein, non-microscopic pores NMP can be used for fixation, such as short term fixation, of resorbable implant 10 to secure resorbable implant 10 in place, such as against a bone surface.

Resorbable implant 10 can comprise any number or shape of non-microscopic pores NMP. As shown for example in FIGS. 1A-4, a number of non-microscopic pores NMP are shown in resorbable implant 10 that are holes that can be defined in and through resorbable implant 10. Non-microscopic pores NMP can extend entirely through resorbable implant 10, can extend only partially through resorbable implant 10, or can do both. Non-microscopic pores NMP can extend, for example, from one outer surface of resorbable implant 10 to an opposite side outer surface. As shown in FIG. 1A-4, for example, non-microscopic pores NMP can extend from surface 12 of resorbable implant 10 all the way through to opposite surface 14. In size, non-microscopic pores NMP can all be the same or of varying non-microscopic diameters. Since resorbable implant 10 can be made with up to, for example, a 99% porosity of microscopic pores MP, creation of non-microscopic pores NMP through resorbable implant 10 can easily intersect many of microscopic pores MP.

Referring to FIG. 5 of the drawings, the bones of a left hand are shown palm side up. The thumb 100 has a first metacarpal bone 102 that connects to the trapezium bone T by forming a mobile joint known as the carpometacarpal (CMC) joint of the thumb. Trapezium T has an articulating surface that contacts another wrist bone which is the scaphoid 104 that forms a joint with the large forearm bone known as the radius bone 106. Trapezium T has other articulating surfaces that come in contact with trapezoid bone 108 and second metacarpal bone 110. The small muscles around first metacarpal 102 at the base of the thumb can rotate around a wide arc in and out of the palm of the hand. The surfaces of trapezium T allow a high degree of mobility of thumb 100. This movement allows thumb 100 to oppose the fingers for pinching and grasping activities.

As noted in the background above, surgical intervention for treatment of CMC arthritis involves removal of the diseased tissue and usually removal the entire trapezium bone or a portion thereof as indicated by arrow A. In prior treatment techniques, a tendon, such as the palmaris longus or flexor carpi radialis, has been harvested from the forearm and rolled up to resemble a rolled “anchovy” wherein the rolled tendon is interposed between the base of first metacarpal 102 and scaphoid 104, the space previously occupied by trapezium bone T.

In accordance with the present disclosure, a resorbable implant can be used to replace all or a portion of a damaged and undesirable bone, such as a trapezium, without any need for tendon grafting and the undesirable consequences associated therewith. FIG. 6 of the drawings shows a view similar to that of FIG. 5 but with trapezium T removed and resorbable implant 10 in its place. After removal of trapezium T, resorbable implant 10 can be provided after it has been formed into a desired configuration, such as a configuration adapted for fitting into the void or space previously occupied by trapezium T. Once in place, resorbable implant 10 can advantageously and optionally be secured in place if desired.

One possible manner of securement of resorbable implant 10 in place is to use sutures or other fastening means that can be routed through at least a portion of non-microscopic pores NMP of resorbable implant 10. Any other suitable securement techniques using non-microscopic pores NMP can be used. FIG. 7 of the drawings shows a close-up view of resorbable implant 10 placed against a surface of a bone B, where bone B can be any bone such as a bone associated with the CMC joint. As shown in this example, sutures S can be used and routed through non-microscopic pores NMP and through holes H that can be drilled into bone B to secure resorbable implant 10 in position against bone B. This securement is considered short-term securement and can prevent migration of resorbable implant 10 while allowing new growth of bone tissue, such as bone tissue BT, into microscopic pores MP of resorbable implant 10. New bone tissue ingrowth can also occur from bone B into non-microscopic pores NMP.

FIG. 8 of the drawings is a cross sectional view of a portion of resorbable implant 10 similar to that in FIG. 7, but illustrating a less close-up view than in FIG. 7. A portion of the surface of resorbable implant 10 can be secured as shown in FIG. 8 against adjacent bone with any suitable fastening mechanism or apparatus, such as for example a resorbable plug 120. As shown in FIG. 8, resorbable plug 120 has been placed through one of the non-microscopic pores NMP and also extends into a hole H that has been created such as by drilling in bone B. Resorbable plug 120 can provide a means of securing resorbable implant 10 in place against bone B for a desirable amount of time, such as an amount of time for new bone tissue to secure and maintain resorbable implant 10 in place.

It will be understood that various details of the presently disclosed subject matter may be changed without departing from the scope of the subject matter. Furthermore, the foregoing description is for the purpose of illustration only, and not for the purpose of limitation.

What is claimed is:

1. A resorbable medical implant comprising:
(a) a resorbable, structural article proportioned in a predetermined shape for placement against a bone surface; and
(b) the structural article comprising a porous material comprising microscopic pores and non-microscopic pores wherein the non-microscopic pores are adapted for use in fixation of the structural article to a bone surface.

2. The resorbable medical implant of claim 1 wherein the structural article is proportioned as and for replacement of a trapezium bone.

3. The resorbable medical implant of claim 1 wherein the structural article comprises a porous material comprising non-microscopic pores that are at least about 0.5 millimeter or greater in size.

4. The resorbable medical implant of claim 3 wherein the structural article comprises a porous material comprising non-microscopic pores that are from about 0.5 to about 2.5 millimeters in size.

5. The resorbable medical implant of claim 1 wherein the structural article comprises a porous material comprising microscopic pores that are less than about 200 microns in size.

6. The resorbable medical implant of claim 1 wherein the structural article comprises a copolymer of lactic acid.

7. The resorbable medical implant of claim 1 wherein the structural article is entirely resorbable.

8. A resorbable medical implant comprising:
(a) a resorbable, structural article proportioned in a predetermined shape for insertion between bone surfaces; and
(b) the structural article comprising a porous material comprising microscopic pores and non-microscopic pores wherein the non-microscopic pores are adapted for use in fixation of the structural article.

9. The resorbable medical implant of claim 8 wherein the structural article is proportioned as and for replacement of a trapezium bone.

10. The resorbable medical implant of claim 8 wherein the structural article comprises a porous material comprising non-microscopic pores that are at least about 0.5 millimeter or greater in size.

11. The resorbable medical implant of claim 10 wherein the structural article comprises a porous material comprising non-microscopic pores that are from about 0.5 to about 2.5 millimeters in size.

12. The resorbable medical implant of claim 8 wherein the structural article comprises a porous material comprising microscopic pores that are less than about 200 microns in size.

13. The resorbable medical implant of claim 8 wherein the structural article comprises a copolymer of lactic acid.

14. The resorbable medical implant of claim 8 wherein the structural article is entirely resorbable.

15. A resorbable medical implant comprising:
(a) a resorbable, structural article proportioned for insertion between adjacent ends of adjacent bones, the structural article being adapted for cushioning the adjacent ends of the adjacent bones;
and
(b) the structural article comprising a porous material comprising microscopic pores of a size less than about 200 microns and non-microscopic pores of a size from about 0.5 to about 2.5 millimeters wherein the non-microscopic pores are adapted for use in fixation of the structural article.

16. The resorbable medical implant of claim 15 wherein the structural article comprises a copolymer of lactic acid.

17. A method of using a resorbable medical implant comprising:
(a) providing a resorbable medical implant comprising:
(i) a resorbable, structural article formed into a predetermined configuration for placement against at least one bone surface; and
(ii) the structural article comprising a porous material comprising microscopic pores and non-microscopic pores wherein the non-microscopic pores are adapted for use in fixation of the structural article; and
(b) placing the resorbable medical implant against a bone surface.

18. The method of claim 17 wherein the structural article comprises a porous material with non-microscopic pores at least about 0.5 millimeter or greater in size and further comprising fixating the resorbable medical implant to the bone surface using the non-microscopic pores of the structural article.

19. A method of using a resorbable medical implant comprising:
(a) providing a resorbable medical implant comprising:
(i) a resorbable, structural article formed into a predetermined configuration for insertion between adjacent ends of adjacent bones, the structural article being adapted for cushioning the adjacent ends of the adjacent bones; and
(ii) the structural article comprising a porous material comprising microscopic pores of a size less than about 200 microns and non-microscopic pores of a size at least about 0.5 millimeters or greater; and
(b) placing the resorbable medical implant into a space between adjacent ends of two bones for cushioning the adjacent ends of the two bones.

20. A method of using a resorbable medical implant comprising:
(a) providing a resorbable medical implant comprising:
(i) a resorbable, structural article proportioned for insertion between adjacent ends of adjacent bones, the structural article being adapted for cushioning the adjacent ends of the adjacent bones; and
(ii) the structural article comprising a porous material comprising microscopic pores and non-microscopic pores wherein the non-microscopic pores are adapted for use in short-term fixation of the structural article;
(b) placing the resorbable medical implant into a space between adjacent ends of two bones for cushioning the adjacent ends of the two bones; and
(c) fixating the resorbable medical implant to at least one of the adjacent bones using the non-microscopic pores of the structural article.
